(19) United States
(12) Patent Application Publication
(10) Pub. No.: US 2009/0240249 A1
(43) Pub. Date: Sep. 24, 2009

(54) System and Method for Performing Ablation and Other Medical Procedures Using An Electrode Array with Flexible Circuit

(75) Inventors: Eric K.Y. Chan, San Carlos, CA (US); Yuri Belman, Campbell, CA (US)

Correspondence Address: FITCH EVEN TABIN & FLANNERY, 120 SOUTH LASALLE STREET, SUITE 1600, CHICAGO, IL, 60603-3406, US

(73) Assignee: Cardima, Inc., Fremont, CA (US)

(21) Appl. No.: 12/476,832

(22) Filed: Jun. 2, 2009

Related U.S. Application Data
(63) Continuation-in-part of application No. 11/268,941, filed on Nov. 8, 2005.
(60) Provisional application No. 60/625,859, filed on Nov. 8, 2004.

(51) Int. Cl. A61B 18/14

(52) U.S. Cl. 606/41

(57) ABSTRACT

A probe for use in medical procedures includes a longitudinal member, a flexible sheath, and a flexible circuit. The longitudinal member includes at least one electrode and at least one thermocouple disposed thereon. The flexible sheath is coupled to and at least partially surrounding the longitudinal member. The flexible circuit is coupled to the sheath and also to the at least one electrode and the at least one thermocouple. The flexible circuit is configured to provide power to the at least one electrode and a return path to the at least one thermocouple.

### CROSS REFERENCES TO RELATED APPLICATIONS

The application is a continuation-in-part of application Ser. No. 11/268,941, filed Nov. 8, 2005, entitled “System and Method for Performing Ablation and Other Medical Procedures using an Electrode Array with Flex Circuit” which claims priority to U.S. Provisional Application Ser. No. 60/625,859 entitled “System and Method for Performing Ablation and Other Medical Procedures using an Electrode Array with Flex Circuit” and filed Nov. 8, 2004, the contents of both of which are incorporated by reference herein in their entireties.

### FIELD OF THE INVENTION

The invention relates to catheters and other medical probes and, more specifically, to using flexible circuits in these devices.

### BACKGROUND OF THE INVENTION

Certain catheters or surgical probe shafts employ a set of braided insulated copper wires that form an intertwined, complicated cross-hatched design running the length of the catheter or probe. This braided shaft then serves as a conduit for radio frequency (RF) current that is delivered to electrodes to ablate tissue, as well as to sense electrophysiological signals that are in turn transmitted along those same lines to a monitoring system.

Another pair of copper wires is often soldered to a copper-constantan thermocouple junction located on a gold band proximal to each electrode. This gold band has a high thermal conductivity and the thermocouple junction quickly equilibrates to the sensed environmental temperature at the gold band. The thermocouple junction forms a temperature-to-voltage transducer and the two copper wires transmit information back to the energy source for feedback-control of RF energy delivery.

Material and labor costs may increase in the assembly process as the number of electrodes increases with conventional methods of assembly. For example, the number of braided wires for a 24-electrode catheter/probe with 24 thermocouples adds up to 72 wires. The “count and cut” process used during assembly to extricate and expose the correct wire along the shaft to solder onto an electrode or thermocouple has become increasingly time-consuming when performing these labor-intensive production steps. Additionally, when one electrode or one thermocouple connection fails during final electrical testing at the factory, the entire catheter/probe has to be counted as scrap if the fault cannot be reworked.

### SUMMARY OF THE INVENTION

A probe is constructed that uses a flexible circuit that is embedded or otherwise attached to a sheath. Modular construction is used so that parts of the probe can be easily replaced or changed. The use of braided wires is eliminated leading to a simplified assembly process, reduced manufacturing costs, and greater user satisfaction with use of the device.

In many of these approaches a probe for use in medical procedures includes a longitudinal member, a flexible sheath, and a flexible circuit. The longitudinal member includes at least one electrode and at least one thermocouple disposed thereon. The flexible sheath is coupled to and at least partially surrounds the longitudinal member. The flexible circuit is coupled to the sheath and is coupled to the electrode and the thermocouple. The flexible circuit is configured to provide power to the electrode and a return path to the thermocouple. The probe may further include connector pins that couple the flexible circuit to the at least one electrode and the at least one thermocouple.

Various materials can be used to construct the sheath. In one example, the sheath can be constructed from silicon. Other examples of materials may also be used. The material selected allows for flexibility in movement of the probe but also is of sufficient strength to allow for maneuvering the probe and for protecting the inner components housed in the sheath.

In many of these examples, the probe includes a proximal end and a distal end and the flexible circuit includes circuit traces adapted to be attached to a terminal connector. The terminal connector is positioned at the distal end of the probe. In other examples, a plurality of electrodes and thermocouples are used and each of the plurality of electrodes is juxtaposed between a selected two of the plurality of thermocouples.

In others of these embodiments, a probe for use in medical procedures includes a longitudinal member, a flexible sheath and a flexible circuit. The longitudinal member has a plurality of coiled electrodes and a plurality of thermocouples disposed longitudinally thereon. Each of the plurality of coiled electrodes are spaced between a selected two of the plurality of thermocouples. The flexible sheath is embedded to and at least partially surrounds the longitudinal member. The flexible circuit is coupled to the sheath and is also coupled to the plurality of electrodes and the plurality of thermocouples. The flexible circuit is configured to provide power to the at least one electrode and a return path to the at least one thermocouple.

Thus, the present approaches allow for the replacement of complex braided wire arrangements with a flexible circuit arrangement. The structures described herein are simple to construct and easy to modify when adjustments are needed and/or when failures of components occur after the flexible circuit assembly is placed inside the sheath.

In addition, the approaches described herein are useful in a variety of medical therapy applications. For instance, the embodiments described herein can also be employed for the treatment of cardiac arrhythmias such as atrial fibrillation (AF) and ventricular tachycardia (VT). Minimally invasive access or endocardial access methods can be employed with probes/catheters using these approaches. The electrodes described herein can also be used to sense electrical activity from the heart, and the proximal connection of the probe/catheter shaft can be attached to a computerized mapping system. In addition, the present approaches are useful in other tissue desiccation and ablation procedures, for example, in oncology to selectively heat and destroy cancerous tumors. Other uses in different organ systems are possible.

### BRIEF DESCRIPTION OF THE DRAWINGS

FIG. 1a is perspective view of a flexible circuit assembly for use in an ablation catheter showing a single electrode thermocouple pair according to the present invention;

FIG. 1b is a front view of a flexible circuit of FIG. 1 showing twenty four electrode-thermocouple pairings according to the present invention;

FIG. 1c is a perspective view showing a three-layered flexible circuit assembly according to the present invention;

FIG. 2a is a perspective view of a flexible circuit assembly with etched electrodes and thermocouples formed into a cylinder according to the present invention;

FIG. 2b is a perspective view of a flexible circuit assembly with coiled electrodes and thermocouples formed into a cylinder according to the present invention;

FIG. 3a is a perspective view of a flexible circuit assembly with etched electrodes and thermocouples formed into a cylinder according to the present invention;

FIG. 3b is a perspective view of a flexible circuit assembly with coiled electrodes and thermocouples formed into a cylinder according to the present invention;

FIG. 4 is a perspective view of a flexible circuit assembly fitted into an ablation catheter according to the present invention; and

FIG. 5 is a cross-sectional view taken along line 304 of FIG. 3a according to the present invention;

FIGS. 6a-c are cross-sectional views of a catheter using three flexible circuit layers according to the present invention;

FIG. 7 is a perspective view of the catheter using three flexible circuit layers of FIG. 6 according to the present invention;

FIG. 8 is perspective view of a flexible circuit sheet showing the electrodes etched directly onto a conductive sheet according to the present invention;

FIG. 9A is a cutaway side view of a flexible circuit assembly housed in a sheath according to the present invention;

FIG. 9B is a bottom view of the flexible circuit assembly of FIG. 9A according to the present invention;

FIG. 9C is a cross-sectional view taken along line “A” of the flexible circuit assembly of FIG. 9A with the flexible circuit attached to the sheath according to the present invention;

FIG. 9D is a cross-sectional view taken along line “A” of the flexible circuit assembly of FIG. 9A with the flexible circuit embedded into the sheath according to the present invention;

FIG. 9E is a cross-sectional view of another example of a flexible circuit assembly according to various embodiments of the present invention;

FIG. 10 is another example of a flexible circuit configuration according to the present invention;

FIG. 11 is an example of configuring a flexible circuit so as to provide flexibility for the circuit according to the present invention;

FIG. 12 is an example of a connector used in the catheters described herein according to the present invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful in a commercially feasible embodiment are often not depicted in order to facilitate a less obstructed view of the various embodiments of the present invention.

### DETAILED DESCRIPTION OF THE PREFERRED EMBODIMENT

The present system and method allows for the replacement of complex braided wire arrangements with a flexible circuit arrangement in catheters and other medical devices. Medical devices constructed according to these approaches are relatively simple to fabricate. Mass production time and costs are also reduced.

The approaches described herein can be used in a variety of medical procedures. For example, the approaches described herein can be employed for the treatment of cardiac arrhythmias such as atrial fibrillation (AF) and ventricular tachycardia (VT). Minimally invasive access or endocardial access methods can also be performed with the probes/catheters described in this application. The electrodes utilized in the approaches described herein can also be used to sense electrical activity from the heart, and the proximal connection of the probe/catheter shaft can be attached to a computerized mapping system. In addition, these approaches can be used in tissue desiccation and ablation procedures, for example, in oncology, to selectively destroy cancerous tumors.

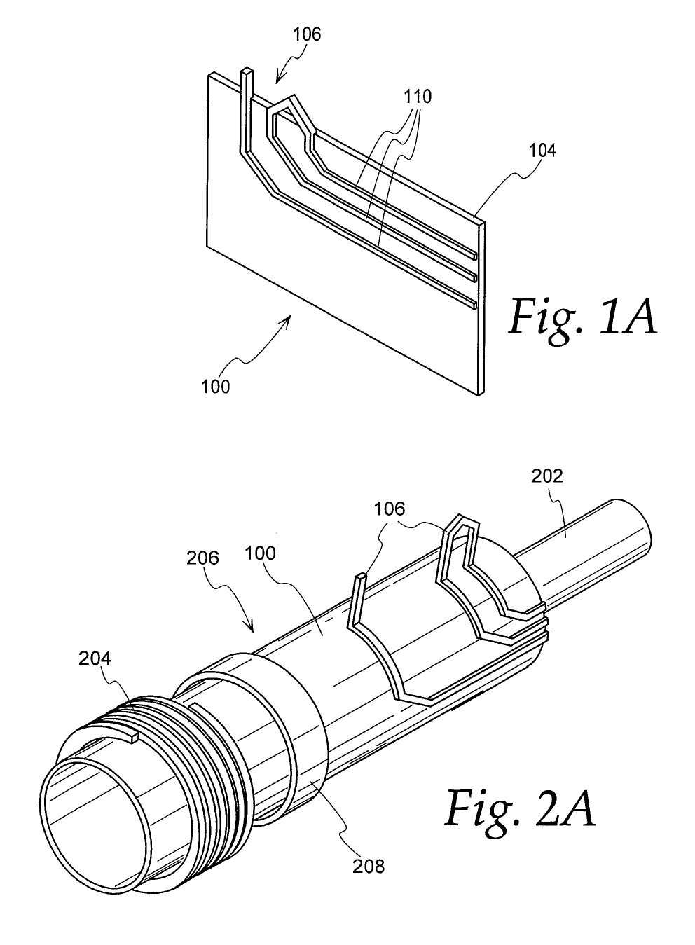

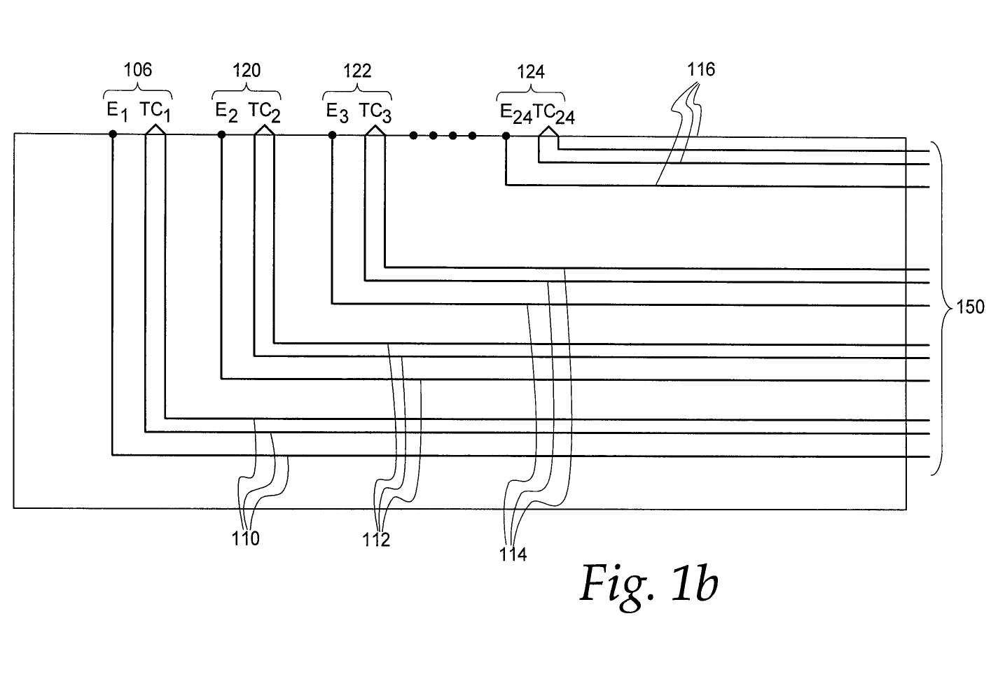

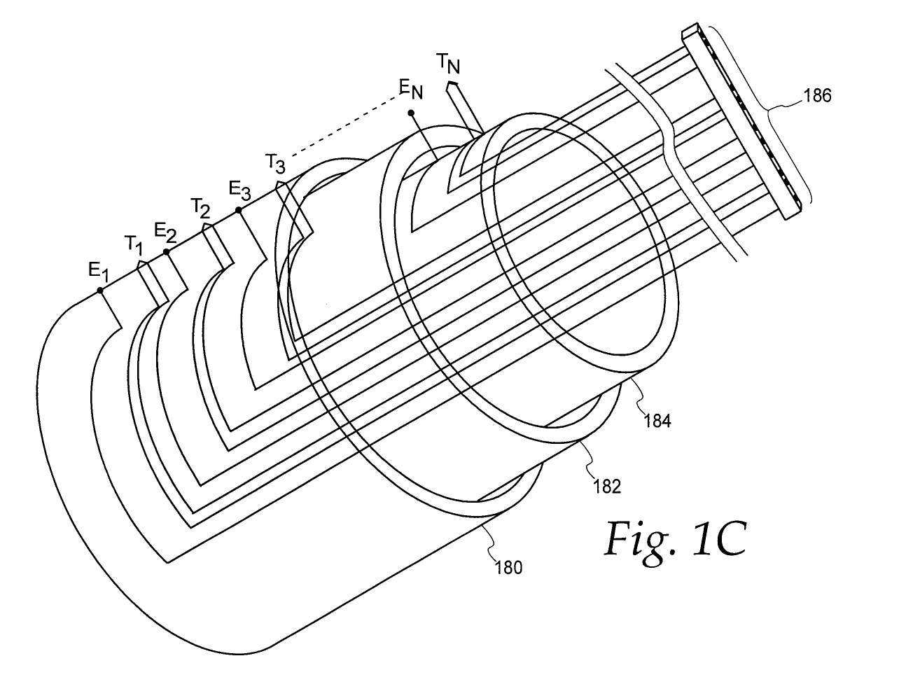

Referring now to FIG. 1a, one example of a flexible circuit 100 used in an ablation catheter is described. A flexible circuit pattern is printed on a flat sheet 104 with solder pins 106 at one edge of the sheet 104. The pins 106 point perpendicular to the surface of the sheet 104. The pins 106 correspond to connections for electrodes and thermal sensing elements (e.g., thermocouples). The pins are shown in FIG. 1 as being parallel to the surface of the sheet 104, but are bent or formed perpendicular to the sheet when the sheet is folded into a cylinder. The following description is made with respect to the thermal sensing elements being thermocouples. However, it will be understood by those skilled in the art that the thermal sensing elements may include not only thermocouples, but thermistors or any other thermal sensing device.

A conductive circuit 110 is established on the pattern and is connected to the pins 106. For example, a metallic conductive circuit 110 is established using techniques that are known in the art. In this case, the conductive circuit 110 includes three lines that conduct electrical energy.

In addition, as described with respect to FIGS. 1b and 1c, repeated similar patterns of the conductive circuits can be printed onto flexible circuit boards. In addition, as described below, this arrangement can be formed into a cylinder and placed into the shaft of a catheter or medical probe.

Conducting circuit elements 110 of the sheet 104 are electrically insulated from each other and from the exposed surfaces of the flexible sheet 104. Preferably, the inter-wire spacings for RF energy and current delivery are predetermined to comply with applicable regulatory, EMC and safety compliance standards.

Referring now to FIG. 1b, a circuit including 24 electrode and thermocouple pairs is shown. A first electrode thermocouple pair 106 (electrode E1 and thermocouple TC1) has corresponding conductive paths 110, which couple the electrode and thermocouples to a connector 150 at the proximal end of the catheter. A second electrode thermocouple pair 120 (electrode E2 and thermocouple TC2) has corresponding conductive paths 112, which couple the electrode and thermocouples to the connector 150 at the proximal end of the catheter. A third electrode thermocouple pair 122 (electrode E3 and thermocouple TC3) has corresponding conductive paths 114, which couple the electrode and thermocouples to the connector 150 at the proximal end of the catheter. For simplicity, the fourth through twenty-third pairs of electrodes and thermocouples are not shown in FIG. 1b. Finally, a twenty-fourth electrode thermocouple pair 124 (electrode E24 and thermocouple TC24) has corresponding conductive paths 116, which couple the electrode and thermocouples to the connector 150 at the proximal end of the catheter.

It will be understood that the electrode thermocouple pairs and their conductive paths can be split across multiple layers of circuit boards. In other words, the first eight pairs may be placed on a first flexible circuit board, the second eight pairings on a second flexible circuit board, and the third eight pairings placed on a third flexible circuit board. The three boards are stacked onto each other and then formed into a cylinder. Preferably, the three groupings are offset lengthwise from each other when the three layers are rolled into a cylinder for placement in the catheter.

Referring now to FIG. 1c, a multi-layered flexible circuit assembly is described. A first assembly 180, second assembly 182, and third assembly 184 are formed into concentric cylinders with assembly 180 being the outermost protective layer assembly. Assembly 182 is inside assembly 180 and assembly 184 is inside assemblies 180 and 182. Electrode solder points E1, E2, and E3 are formed on the assembly 180. Other electrode solder points up to and including electrodes En are formed on the other assemblies. The assemblies 180, 182, and 184 are electrically insulated from each other by homogenous polyimide material layers (not shown in FIG. 1c) that are typically used in multi-layer flexible circuit boards.

In addition, thermocouple solder points T1, T2 and T3 are formed on assembly 180. Other thermocouple solder points up to and including Tn are formed on the assemblies 182 and 184. Conductive lines 186 are coupled to the respective electrodes and thermocouples. The electrodes and thermocouples are attached to the actual solder points.

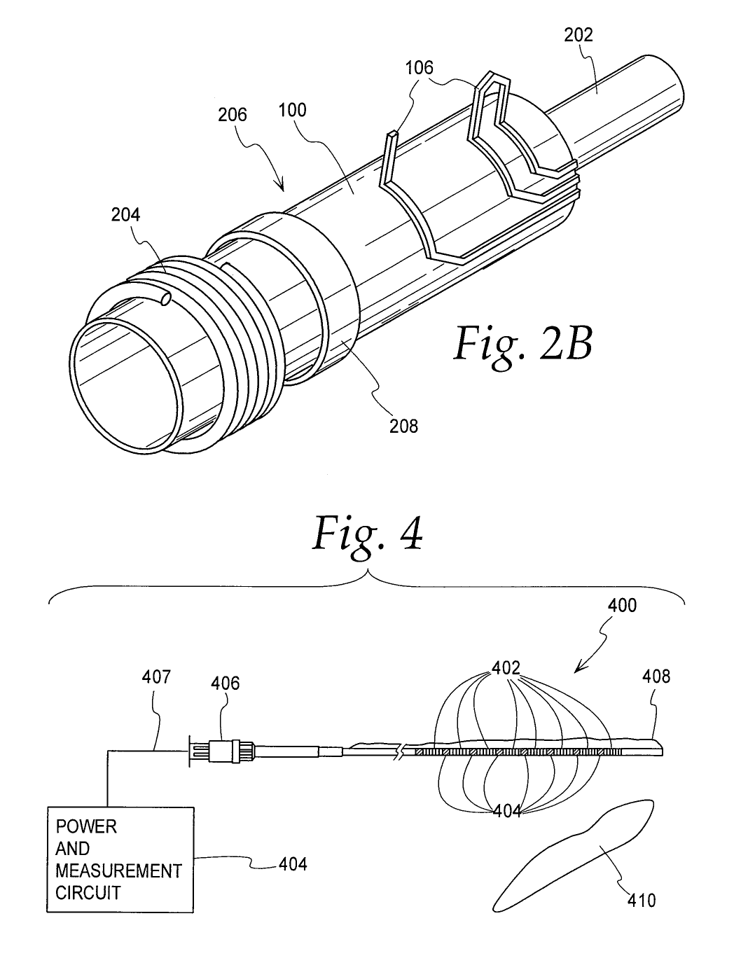

Referring now to FIG. 2a, the flexible sheet 100 is shown folded into a cylinder 206. For example, the flexible sheet 100 may be folded around a shape-forming mandrel 202, with the pins 106 at the sheet edge pointing away from the mandrel 202. In this case, the underside of the edge of the flexible sheet 100 with pins 106 is adhered to the top surface of the other edge of the same sheet 100, so that the sheet takes on a cylindrical form. The pins 106 are soldered onto etched electrodes 204. The pins 106 (shown exaggerated in FIG. 2a for clarity) protrude perpendicularly along one longitudinal edge of the cylinder 206.

A thermocouple band 208 is also constructed. In one example, the thermocouple band 208 may be constructed of a gold band to give the band a high thermal conductivity. These bands can be constructed using techniques known by those skilled in the art.

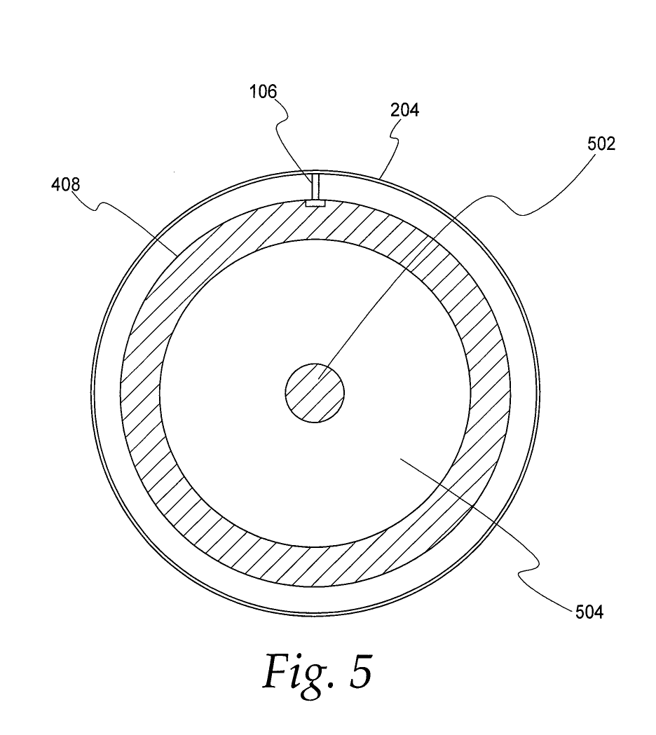

The example described herein with respect to FIG. 2a (and also FIGS. 3a and 5) utilizes a single set of electrodes and thermocouple band. However, multiple electrodes and bands can also be used. It will also be understood that multiples of the unit assembly can be organized in a linear pattern to form a linear mapping and ablation electrode array.

Preferably, metal etching is used for the production of the electrodes 204 to produce coiled groove, thereby creating a spring-like electrode component. Several techniques may be employed to etch metal sheets into different structural forms.

In one example process, a computer-aided design (CAD) drawing of the electrode coil pattern is generated. This drawing serves as the CAD image that is a faithful replica of the electrode. The drawing is printed onto a transparency film.

A cylindrical section of metal (e.g. platinum iridium) cut to a specific length is cleaned thoroughly. Then, a photo resist coating is applied to the outer surface so that it is photo-sensitive.

The CAD image is then overlaid onto the photo-sensitized metal surface and exposed to a ultra-violet (UV) light source. The metal cylinder is thereafter deposited into a developing solution to create a hardened image of the desired coil pattern on the metal cylinder surface.

The metal surface is then treated with an etchant, such as an acid. The etchant eats away the rest of the surface that is devoid of the hardened image, to create a spiral-shaped coil structure that can function as ablation and mapping electrodes 204. If the desired spiral groove is too fine for acid or other form of chemical etching, then an alternate fabrication technique is to employ three dimensional etching of the spiral pattern via a precision laser cutting process.

Yet another alternate process is to etch the electrodes directly onto the flexible circuit board. This approach assumes dissimilar metals are layered onto the board, e.g. platinum for electrodes, copper for conduction lines by an appropriate manufacturing process.

Referring now to FIG. 2b, another example of a flexible circuit assembly is described. In this case, the assembly is the same as that shown and described with respect to FIG. 2a except that the etched electrodes 204 are replaced with coiled electrodes 204.

In one example, the coiled electrodes 204 may be 0.005″ gauge (0.003″ to 0.006″ range with one preferred type being a 0.005″ gauge) platinum iridium wire that is wound into a spring-like structural unit. These units may be 3 mm to 6 mm long and have outer diameters ranging from approximately 3 Fr to 5 Fr. Other dimensions are also possible.

Referring now to FIG. 3a, the etched electrodes 204 and thermocouple band 208 are inserted over the cylindrical structure formed by folding the flexible circuit. The electrodes 204 and thermocouple 208 are soldered at the respective protruding pin sites 106 that were spaced out by design to provide the desired inter-electrode and electrode-thermocouple spacing.

At one stage of the manufacturing process, the electrodes 204 can be coated with a conductive gel or other ionic material that improves tissue-electrode contact. At the same time, the electrodes 204 may be infused with anti-coagulant chemicals that are time released during the course of an ablation procedure.

Multiple layers of such unit assemblies may be utilized to reduce overall catheter or medical probe shaft diameter. These layers can be electrically insulated from each other by a homogenous polyimide material that is typically used in multi-layer flexible circuit boards.

An inner hollow shaft 302 of the resulting cylinder from this flexible circuit catheter shaft can serve as a conduit for a guide wire or stylet with deflectable mechanism, permitting the linear assembly of electrodes 204 and thermocouples 208 to be shaped and conformed to a tissue surface to afford excellent electrode-tissue contact that ensures optimal coupling of RF energy with the tissue. The conductive annular gold band for the thermocouple and the etched electrode are then slid along the shaft and soldered over their respective solder points.

The flexible circuit assembly is rolled and placed in the shaft of the catheter. The end of the flexible circuit assembly plugs into a connector. The connector is coupled to at least one PC card, which interfaces the arrangement to power and measurement equipment.

Referring now to FIG. 3b, another example of a flexible circuit assembly is described. In this case, the assembly is the same as that shown and described with respect to FIG. 3a except that the etched electrodes 204 are replaced with coiled electrodes 204.

As with the coiled electrodes of FIG. 2a, the coiled electrodes 204 of FIG. 3b may be 0.005″ gauge (0.003″ to 0.006″ range with one preferred type being a 0.005″ gauge) platinum iridium wire that is wound into a spring-like structural unit. These units may be 3 mm to 6 mm long and have outer diameters ranging from approximately 3 Fr to 5 Fr. Other dimensions are also possible.

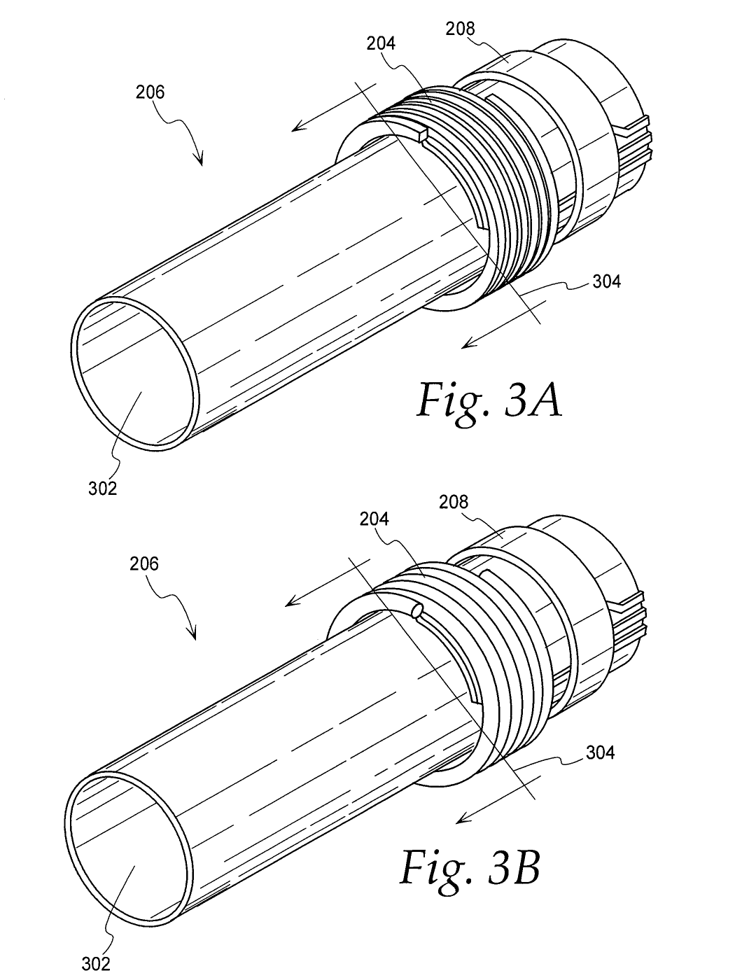

Referring now to FIG. 4, one example of a catheter system using the flexible circuit and etched electrodes and thermocouples is described. A catheter 400 includes the cylindrical flexible circuit assembly 408 that has been described with respect to FIGS. 1-3 above. The cylindrical assembly 408 forms the distal end of the catheter 400 and is inserted into the telescopic structure 406 having a handle, which forms the proximal end of the catheter 400.

Etched electrodes 402 are constructed and soldered onto the cylindrical assembly 408 as has been described elsewhere in the application. Alternatively, coiled electrodes may be used. In addition, thermocouples 404 are soldered onto the cylindrical assembly 408 as has also been described elsewhere in the application. The cylindrical assembly 408 may include sub-portions of flexible circuits that are attached together to form the assembly 408.

An inner hollow shaft (not shown in FIG. 4) of the cylinder 408 (i.e., the flexible circuit catheter shaft) may serve as a conduit for a guide wire or stylet with deflectable mechanism (not shown), permitting the linear assembly of electrodes 402 and thermocouples 404 to be shaped and conform to a tissue surface. This gives excellent electrode-tissue contact that ensures optimal coupling of RF energy with tissue 410. The conductive annular gold band for the thermocouples 404 and the etched electrode 402 may then be slid along the shaft and soldered over their respective solder points.

A power and measurement circuit 408 is coupled to the catheter 400 via a personal computer (PC) board 407. The power and measurement circuit 408 supplies electrical energy to the catheter and its electrodes 402 that can be used, for example, for ablation procedures. The impedance signals received at the electrodes and the information received by the thermocouples reporting tissue temperature can be relayed back to the power and measurement circuit 408 via the cylindrical assembly 408. The power and measurement circuit 408 can receive information from the thermocouples and display this information to an operator for manual feedback control. In addition, the power and measurement circuit 408 can receive operating instructions from an automated processing unit for feedback and control to adjust various operating parameters pertaining to the RF current being emitted from the catheter 400, such as the power or current delivered to the tissue 410.

Referring now to FIG. 5, a cross-sectional view of the cylindrical assembly 208 taken along line 304 in FIG. 3a is described. A guide wire 502 is in the middle of the hollow shaft 504 of the assembly 408. The electrodes 204 and thermocouple (not shown in FIG. 5) are soldered at the respective protruding pin sites 106 that were spaced at predetermined distances by design to provide the desired inter-electrode and electrode-thermocouple spacing along the side of the catheter.

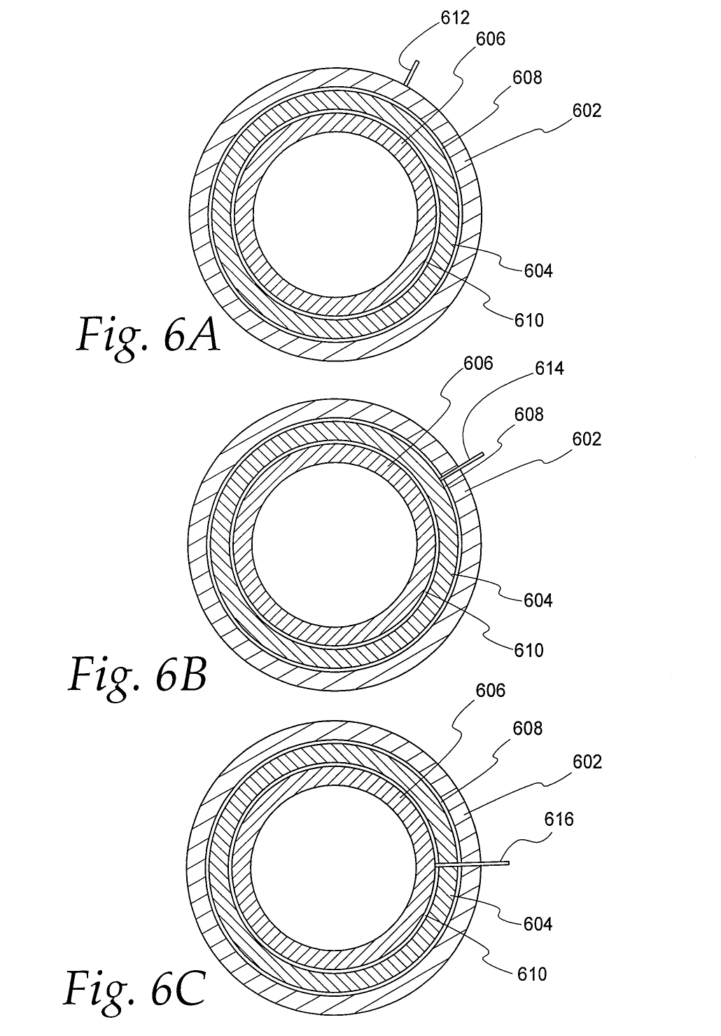

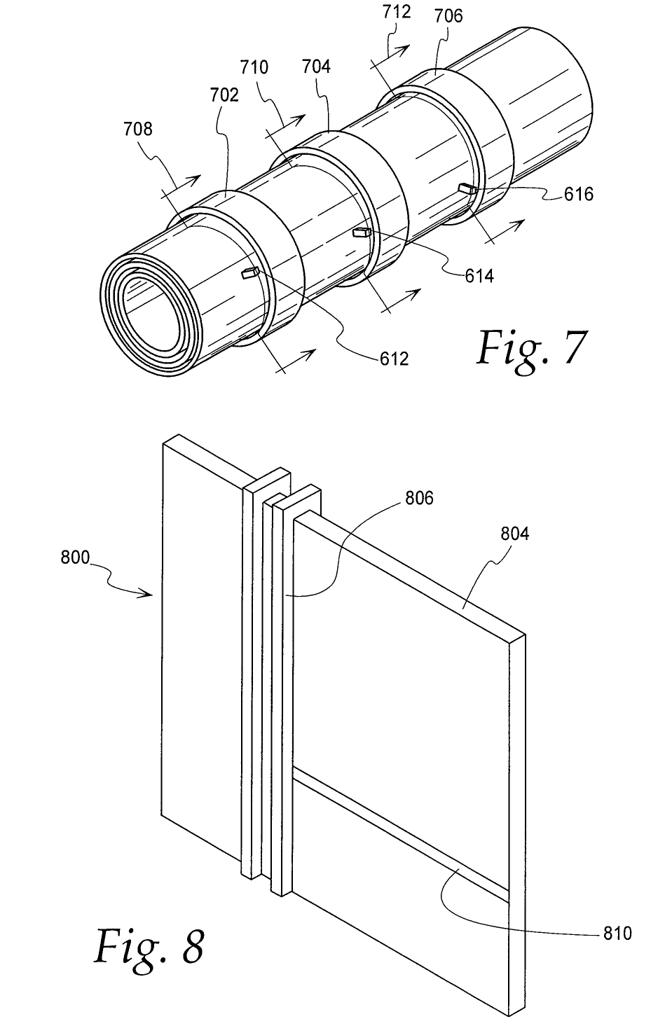

Referring now to FIG. 6a-c and FIG. 7, one example of an assembly using multiple layers of flexible circuits is described. FIGS. 6a-c show cross sectional drawings taken along lines 708, 710, and 712 of FIG. 7 respectively. A first flexible circuit assembly 602, second flexible circuit assembly 604, and third flexible circuit assembly 606 are concentrically located with assembly 602 on the outside, assembly 604 inside of assembly 602 and assembly 606 inside assembly 604.

The assemblies 602, 604, and 606 are electrically insulated from each other by a homogenous polyimide material layers 608 and 610 that is typically used in multi-layer flexible circuit boards. Pin 612 is coupled to the flexible circuit assembly 602. Pin 614 extends through the assembly 602 and is coupled to the flexible circuit assembly 604. Pin 616 extends through the assemblies 602 and 604 and is coupled to the flexible circuit assembly 606. Although only one pin is shown for each assembly (for convenience in viewing), it will be understood that multiple pins for the multiple layers 602, 604, and 606 can be used. In addition, additional pins for thermocouples may also be included. The inner pins 614 and 616 may have holes drilled through the various layers so that the pins 614 and 616 reach above the surface of the cylinder.

Referring now to specifically to FIG. 7, the assembly of FIG. 6 shows electrodes and thermocouples 702 coupled to the pins 612. Electrodes and thermocouples 704 are coupled to the pins 614. Further, electrodes and thermocouples 706 are coupled to the pins 616. Since multiple layers are used, the overall catheter or medical probe shaft diameter is reduced.

Referring now to FIG. 8, one example of a flexible circuit 800 used in an ablation catheter is described where the electrodes are etched directly onto the flexible sheet. A flexible circuit pattern is printed on a flat sheet 804. Electrodes 806 are constructed on the sheet 804 directly and electrically contact a conductive circuit element 810 on the flexible sheet 804. Dissimilar metals are layered onto the board, for instance, platinum for electrodes and copper for the conduction circuit element 810, by an appropriate manufacturing process.

Conductive circuit elements 810 of the sheet 804 are electrically insulated from each other and from the exposed surfaces of the flexible sheet 804. Preferably, the inter-wire spacings for RF energy and current delivery are predetermined to comply with applicable regulatory, EMC and safety compliance standards.

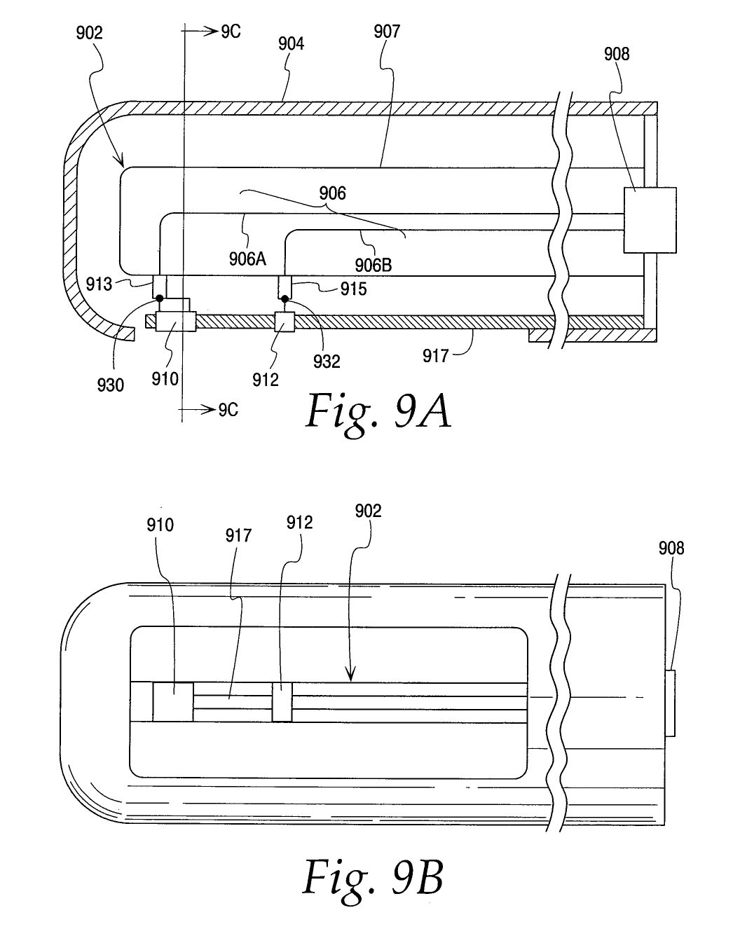

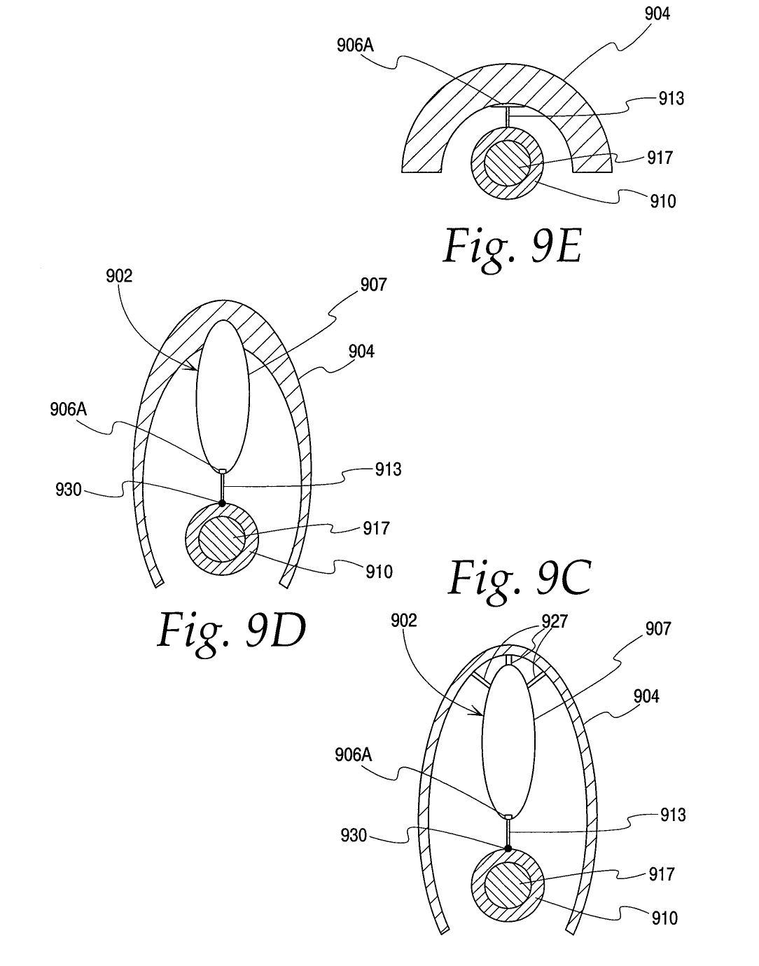

Referring now to FIGS. 9A-D, one example of a flexible circuit arrangement 900 including a sheath 904 is described. As shown, a flexible circuit 902 is embedded in or otherwise attached to the sheath 904. If the flexible circuit 902 is embedded in the sheath 904 (as shown in FIG. 9D), the flexible circuit 902 may be inserted into and surrounded by some portions of the sheath 904 such that the sheath 904 secures the flexible circuit 902 in place. In some of these examples, the flexible circuit 902 may be formed so as to be coextensive with the sheath 904.

If the flexible circuit 902 is attached to the sheath 904 (as shown in FIG. 9C), any type of fastening arrangement (e.g., glue, screws, nails, rivets, ultrasonic welding to name a few examples) may be used to secure the two elements together. In the example of FIG. 9C, the flexible circuit 902 is attached to the sheath 904 by fasteners 927. In this example, the fasteners 927 are bolts or the like. The flexible circuit 902 includes a sheet 907 and circuit traces 906. The sheet 907 may be made of a material such as Kapton to give one example.

The sheath 904 can be constructed from silicon, silicone rubber, or some other suitable material. The sheath 904 is flexible and as described herein houses the electrically conductive elements of the system. In addition, the sheath 904 is constructed and configured to meet governmental and non-governmental compliance standards concerning, for example, the conduction of RF current and other operating characteristics.

In many examples, the sheath 904 is constructed of materials that provide strength for the assembly and allow the assembly to be moved by the user. On the other hand, the materials are not so rigid as to hamper the movement and maneuverability of the assembly 900 when used as intended during a given medical procedure. The sheath 904 also provides protection for the components it houses, for example, from accidental damage caused by misuse of the assembly 900.

In some examples, the sheath 904 is formed over only the distal end of a catheter assembly. In other examples, the sheath 904 may be formed over the entire length of the catheter assembly. It will be appreciated that the dimensions, shape, and extent of the sheath 904 may be varied according to the requirements of the system and the application.

The trace elements 906 include a first trace line 906a and a second trace line 906b and these lines extend to a connector 908. The connector 908 allows electrical connection to external systems. For simplicity, the example of FIGS. 9A-D, show two trace lines 906a and 906b on the sheet 907. The sheet 907 may be any type of circuit board used to contain electrical trace elements. The first trace line 906a is associated with an electrode 910 and the second trace line 906b is associated with a thermocouple 912. It will be appreciated that any number of trace lines can be used that correspond with any number of electrodes and thermocouples. It will also be understood that the sheet 907 can also be configured (e.g., rolled) according to any of the configurations described elsewhere herein.

It will further be appreciated that the electrodes and thermocouples associated with the flex circuit 902 are often used in pairs. However, it will also be understood that in some situations only a single element (e.g., a single electrode) may be used. In other examples, single electrodes may be matched with multiple thermocouples and in still other examples, single thermocouples may be matched with multiple electrodes.

The connector 908 may be coupled to an external power generating and monitoring system such as the INTELLITEMP® system manufactured by Cardima, Inc. In one example, the connector 908 is a plug-in type connector that plugs into an external power generating and monitoring system. Other types of connectors may also be used. The trace line 906a is connected to the electrode 910 via a first pin 911 at a first soldering point 930 and the trace line 906b is attached to the thermocouple 912 via a second pin 913 at a second soldering point 932.

The trace elements 906 may be printed on the flat sheet of material 907 with the pins 911 and 913 being soldered at one edge of the sheet. The pins 911 and 913 themselves may extend perpendicular to the surface of the sheet 907. However, in other examples, the pins 911 and 913 are bent when the sheet 907 is folded into a cylinder.

The pins 911 and 913 mate with/are attached to proximal exit points of the conducting trace elements 906 and these pins are any type of suitable medical grade terminal connectors. One pin 911 is soldered to solder points 930 of the electrode 910 and the other pin 913 is soldered to solder point 932 of the thermocouple 912 to form connections between the electrode 910 and the trace line 906a, and the thermocouple 912 and the trace line 906b respectively.

The trace elements 906 form a conductive circuit that transmits electrical energy that is established on the sheet 907 and is connected to the pins 913 and 915. For example, the trace elements 906 are established on the sheet 907 using techniques (e.g., circuit printing) that are known in the art.

The trace elements 906 are electrically insulated from each other on the sheet 907 and are electrically insulated from the exposed surfaces of the sheet 907. The inter-wire spacing of trace line 906a and trace line 906b for RF energy and current delivery are selected to comply with applicable regulatory, EMC and safety compliance standards.

The electrode 910 and the thermocouple 912 are assembled along a longitudinal member 917 (e.g., a hollow polymer tube having a suitable diameter). The longitudinal assembly 917 is constructed from flexible and/or pliable materials and is securely fastened (e.g., by glue) or otherwise fastened by any type of fastening arrangement (e.g., screws, rivets, ultrasonic welding to name a few examples) to the interior of the sheath 904.

It will be understood that the electrode and thermocouple pairs and their conductive paths can be split across multiple sheets or circuit boards. In other words and as describe elsewhere herein, first grouping of electrode/thermocouple pairs may be placed on a first flexible circuit board, a second grouping of the pairs on a second flexible circuit board, and a third grouping of the pairs placed on a third flexible circuit board. The three boards are stacked onto each other and then formed into a cylinder. Preferably, the three groupings are offset lengthwise from each other when the three layers are rolled into a cylinder for placement in the catheter.

The thermocouple 912 (e.g., a thermocouple band) may be constructed of various materials. In one example, the thermocouple band 912 may be constructed of gold give the band a high thermal conductivity. These bands can be constructed using techniques known by those skilled in the art. The thermocouple 912 provides temperature information that is returned to an external device (e.g., the INTELLITEMP® system manufactured by Cardima, Inc.) via the trace elements of flexible circuit 902, which act as a return path.

Various techniques can be used to construct the electrode 910. In one example and as described elsewhere herein, metal etching is used for the production of the electrode 910 to produce a coiled groove, thereby creating a spring-like electrode component. The etching process allows precisely-dimensioned parts to be constructed with precise tolerances. Various techniques may be employed to etch metal sheets into different structural forms. Alternatively, a coiled electrode that is not produced using an etching process may also be used.

In one example of constructing the assembly 900, the sheath 904 is initially and separately formed and then the flexible circuit 902 is embedded or otherwise attached to the sheath 904. In another example, the sheath 904 is formed together with the flexible circuit 902. Various manufacturing techniques and tools may be used to form these elements that are well known to those skilled in the art. The connector pins 913 and 915 may be formed with the flexible circuit 902 or formed separately and then attached to the flexible circuit 902.

The electrode 910 and thermocouple 912 are attached to the longitudinal member 917 by gluing, soldering, or some other fastening approach. Then, the connector pins 913 and 915 (on the flexible circuit 902) are aligned to the electrode soldering point 930 (on the electrode 910) and the thermocouple soldering point 932 (on the thermocouple 912). The electrode 910 and the thermocouple 912 are soldered to the pins 913 and 915 and the assembly is complete.

Referring now to FIG. 9E, another example of a catheter structure is described. In this example, the trace elements 906A are embedded in the sheath 904. Consequently, the flexible circuit 902 is not used in this example. In other examples, some of the trace elements are embedded in the sheath while others are present on a flexible circuit board.

Consequently, the need to use and construct a braided wire assembly as used in previous approaches is eliminated. More specifically, there is no need to “count and cut” the braided wires and thread the wires through the assembled as required in previous approaches. In other words, the prefabricated silicon sheath with embedded flexible circuit takes on the functions of the braided wires and the associated problems with maneuvering these wires are eliminated. Consequently, manufacturing and assembly costs are reduced due to the ease and speed by which the assembly 900 can be constructed.

As can also be appreciated, the assembly 900 is modular in structure. Various parts of the assembly 900 can be replaced without disassembling the entire structure. For example, the pins, electrodes, and thermocouples can be removed easily and quickly in contrast to previous systems where the entire assembly needed to be disassembled to replace a malfunctioning part. Further, the flexible circuit boards themselves can also be easily removed and replaced in the present approaches thereby obviating the need for removing braided wires and avoiding the problems associated with rewiring the system. Potentially, during the assembly process, a technician can easily replace damaged parts of the circuit with new flexible circuit components as required using all the present approaches.

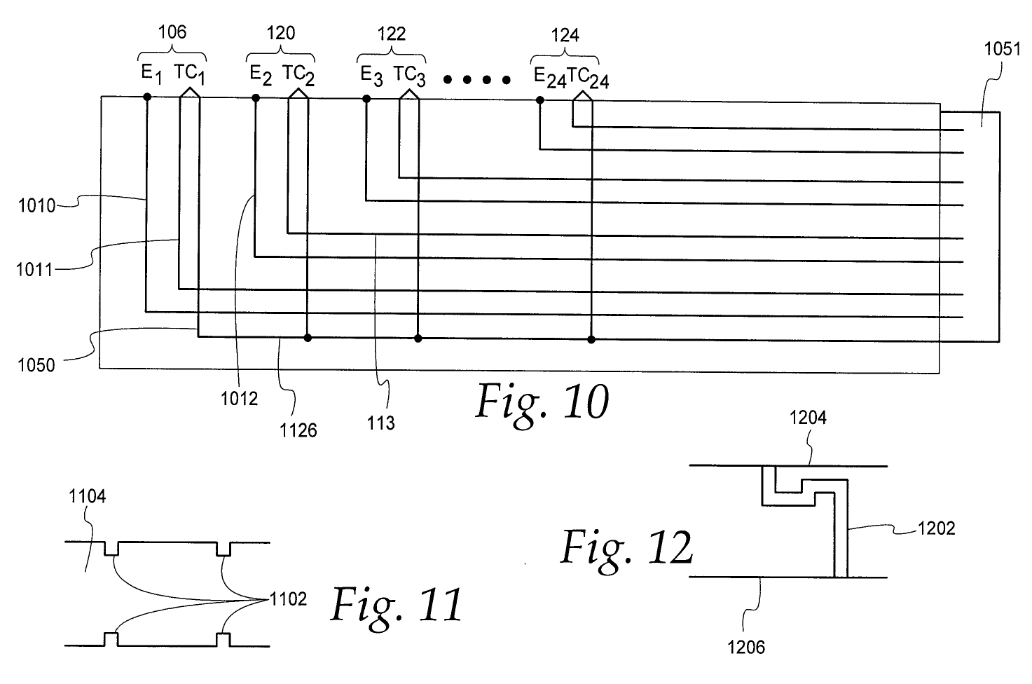

Referring now to FIG. 10, another example of a flexible circuit configuration is described. In this example, the thermocouples have a constantan lead and a copper lead. However, it will be appreciated that other configurations or materials may be used.

As shown, the circuit includes 24 electrode and thermocouple pairs. A first electrode thermocouple pair 1006 (electrode E1 and thermocouple TC1) has corresponding conductive path 1010 for the electrode, and a conductive path 1011 coupled to the constantan lead of TC1. The copper lead of the thermocouple TC1 is coupled to a common path 1050 (e.g., common copper path). The conductive paths 1010, 1011, and 1050 couple the electrode and thermocouples to a connector 1051 at the proximal end of the catheter.

A second electrode thermocouple pair 1020 (electrode E2 and thermocouple TC2) has a corresponding conductive path 1012 to couple the electrode E2 to the proximal end of the catheter. A conductive path 1013 couples the constantan lead of TC2 to the connector 1051. The copper lead of the thermocouple TC2 is coupled to the connector 1051 via the common copper conductor 1050. Similarly, a third electrode thermocouple pair 1022 (electrode E3 and thermocouple TC3) up through a twenty-fourth electrode thermocouple pair 1024 (electrode E24 and thermocouple TC24) are connected to the connector 1051 in a similar manner. It will be appreciated that the use of a common conductor 1050 eliminates the need for 23 trace lines thereby reducing the size of the catheter assembly.

In another example, mechanical flexibility can be provided to the flexible circuit structure by adding cuts in this structure. As shown in FIG. 11, cuts 1102 are added to the flexible circuit 1104 in a longitudinal direction of the circuit 1104 as it is positioned in the catheter (e.g., in the sheath). This allows flexibility by allowing the structure to bend and/or move back and forth. The dimensions and frequency of the cuts along the circuit 1104 may be varied according to the requirements of the system.

In other examples and now referring to FIG. 12, a connector pin 1202 connects a flexible circuit 1204 and an electrode 1206. The pin 1202 is configured in an “S”-like shape and is constructed from a material that provides at least some bounce (between the flexible circuit and electrode or other components) and helps absorb shocks that the catheter may experience during use. It will be appreciated that the exact dimensions and shape of the pin 1202 may vary depending upon the requirements of the system.

While there has been illustrated and described particular embodiments of the present invention, it will be appreciated that numerous changes and modifications will occur to those skilled in the art, and it is intended in the appended claims to cover all those changes and modifications which fall within the true scope of the present invention.

What is claimed is:

1. A probe for use in medical procedures, comprising:
a longitudinal member that includes at least one electrode and at least one thermocouple disposed thereon;
a flexible sheath coupled to and at least partially surrounding the longitudinal member;
a flexible circuit physically coupled to the sheath and being coupled to the at least one electrode and the at least one thermocouple, the flexible circuit configured to provide power to the at least one electrode and a return path to the at least one thermocouple.

2. The probe of claim 1 further comprising connector pins that couple the flexible circuit to the at least one electrode and the at least one thermocouple.

3. The probe of claim 2 wherein the connector pins comprise pins that are configured to provide a bounce between the flexible circuit and the at least one electrode.

4. The probe of claim 1 where the sheath is comprised of silicon.

5. The probe of claim 1 where the flexible circuit comprises a single member that extends along a longitudinal axis of the sheath.

6. The probe of claim 1 wherein the longitudinal member is constructed of a flexible material.

7. The probe of claim 1 wherein the probe includes a proximal end and a distal end and wherein the flexible circuit comprises circuit traces adapted to be attached to a terminal connector, the terminal connector positioned at the distal end of the probe.

8. The probe of claim 7 wherein at least some of the traces are embedded in the sheath.

9. The probe of claim 7 wherein the traces comprise a common conductor for each of the thermocouples.

10. The probe of claim 1 wherein the at least one electrode comprises a plurality of electrodes and wherein the at least one thermocouple comprises a plurality of thermocouples and each of the plurality of electrodes is juxtaposed between a selected two of the plurality of thermocouples.

11. The probe of claim 1 wherein the at least one electrode and at least one thermocouple are disengagably attached to the flexible circuit.

12. A probe for use in medical procedures, comprising:
a longitudinal member having a plurality of coiled electrodes and a plurality of thermocouples disposed longitudinally thereon, each of the plurality of coiled electrodes spaced between a selected two of the plurality of thermocouples;
a flexible sheath embedded into and at least partially surrounding the longitudinal member;
a flexible circuit coupled to the sheath and being coupled to the plurality of electrodes and the plurality of thermocouples, the flexible circuit configured to provide power to the at least one electrode and a return path to the at least one thermocouple.

13. The probe of claim 12 further comprising connector pins that couple the flexible circuit to the plurality of electrodes and the plurality of thermocouples.

14. The probe of claim 13 wherein the connector pins comprise pins that are configured to provide a bounce between the flexible circuit and the at least one electrode.

15. The probe of claim 12 where the sheath is comprised of silicon.

16. The probe of claim 12 where the flexible circuit comprises a single member that extends along a longitudinal axis of the sheath.

17. The probe of claim 12 wherein the longitudinal member is constructed of a flexible material.

18. The probe of claim 12 wherein the probe includes a proximal end and a distal end and wherein the flexible circuit comprises circuit traces adapted to be attached to a terminal connector, the terminal connector positioned at the distal end of the probe.
